(19) United States
(12) Patent Application Publication
(10) Pub. No.: US 2026/0351380 A1
(43) Pub. Date: Nov. 5, 2026

(54) SYSTEM AND METHOD TO PREDICT LYMPH NODE STATUS IN PATIENTS WITH CANCER USING PRE-OPERATIVE PARAMETERS AND MACHINE LEARNING

(71) Applicants: Jashna Oza, San Jose, CA (US); Dushyant Mandlik, Ahmedabad (IN)

(72) Inventors: Jashna Oza, San Jose, CA (US); Dushyant Mandlik, Ahmedabad (IN)

(21) Appl. No.: 19/339,362

(22) Filed: Sep. 25, 2025

Related U.S. Application Data
(63) Continuation-in-part of application No. 19/264,951, filed on Jul. 10, 2025.
(60) Provisional application No. 63/799,585, filed on May 4, 2025.

(51) Int. Cl. G16H 20/40; A61B 5/00; G16H 10/60; G16H 50/20

(52) U.S. Cl. CPC G16H 20/40; A61B 5/742; G16H 10/60; G16H 50/20

(57) ABSTRACT

The present invention provides a system and method for the automated prediction of pathological cancer parameters using only preoperative data, thereby enhancing early decision-making in oncology. By curating and preprocessing clinical, radiological, and non-pathological patient data, the system employs multiple machine learning models to predict key pathological features such as tumor stage (pT), nodal stage (pN), depth of invasion (DOI) etc. The use of a voting-based ensemble mechanism improves accuracy and robustness, with the system generating confidence scores and prediction accuracy statistics for each parameter. Based on the predicted pathological features, the invention further calculates the final pathological cancer stage and assists in determining the need for neck node metastasis surgery. The invention reduces reliance on invasive post-surgical histopathology, enhances clinical efficiency, and supports personalized cancer treatment planning. It is scalable, adaptable to various healthcare systems, and supports federated learning for continuous improvement.

### CROSS REFERENCE TO RELATED APPLICATIONS

This application is a Continuation-In-Part of U.S. patent application Ser. No. 19/264,951, filed on Jul. 10, 2025, which in turn claims the benefit of U.S. Provisional Patent Application No. 63/799,585 filed on May 4, 2025. The contents of the foregoing patent applications are incorporated by reference herein in their entirety.

### TECHNICAL FIELD

The present invention relates to the field of oncology and medical diagnostics, specifically to a system and method to predict lymph node status in patients with cancer using pre-operative parameters and machine learning.

### BACKGROUND OF INVENTION

Accurate estimation of pathological parameters in cancer patients, such as Depth of Invasion (DOI), nodal involvement, and tumor grade, is a critical determinant in formulating treatment strategies and surgical planning. Conventionally, such assessments are performed through invasive biopsy procedures or post-operative histopathological analysis, which inherently introduces delays in treatment initiation and increases patient morbidity. The current preoperative clinical methodology primarily relies on radiological imaging modalities such as MRI, CT, or ultrasound. However, these techniques exhibit inherent measurement inconsistencies due to interpersonal and intrapersonal variations. These inconsistencies arise from interobserver variability, imaging resolution constraints, and tumor heterogeneity, collectively contributing to discrepancies in pathological estimation. Such variability in preoperative assessments affects the reliability of clinical decision-making, potentially leading to suboptimal treatment planning.

For example, in cases of Squamous Cell Carcinoma of the Oral Tongue (SCCOT), cancer tends to spread (metastasize) to the lymph nodes in the neck. This is a significant concern in treatment planning because metastasis to the neck nodes can impact the severity of the cancer, prognosis, and the need for surgical intervention or other therapies. Since SCCOT has a high likelihood of lymph node metastasis, accurate preoperative estimation of pathological parameters, particularly DOI and nodal status, is essential for determining the risk of nodal spread. Reliable prediction of these parameters can assist clinicians in evaluating the necessity of elective neck dissection, thereby optimizing surgical decision making.

One of the major challenges in the field of oncology is the risk of performing unnecessary or delayed surgical procedures. Conventional clinical methodologies lack a standardized, objective, and automated framework capable of integrating radiological and physiological parameters for the preoperative prediction of pathological characteristics. As a result, patients frequently undergo multiple surgical interventions, first for primary tumor excision, and subsequently for additional procedures, such as neck node dissection, based on postoperative pathological analysis. This multi-stage surgical approach contributes to increased patient distress, elevated healthcare costs, and a higher incidence of postoperative complications.

Therefore, there is a need for an objective, automated model that integrates radiological and clinical parameters to predict key pathological features such as DOI and nodal involvement. Ideally, such a model should also incorporate a broader range of patient-specific inputs, including non-pathological and genetic data, to create a truly personalized predictive assessment. Such a system would reduce reliance on subjective imaging and clinical assessments, mitigate variability in clinical judgment, minimize unnecessary surgeries, and enable earlier, more precise intervention, ultimately improving patient outcomes in oncology.

The above-mentioned shortcomings, disadvantages and problems are addressed herein, which will be understood by reading and studying the following specification.

### SUMMARY

Described herein is a system for predicting lymph node metastasis status in patients with cancer using pre-operative parameters and machine learning, the system comprising: a memory 104 storing program instructions; a processor 106 configured to execute the instructions to: acquire a set of pre-operative patient parameters, the parameters comprising clinical data, radiological data, and non-pathological patient data for personalizing a prediction; preprocess the pre-operative patient parameters to generate structured patient-specific data; apply a plurality of machine learning models to the structured patient-specific data, wherein each machine learning model is trained on historical pathological outcomes, to generate pathological predictions; aggregate the pathological predictions from the plurality of machine learning models using a voting mechanism to compute final predicted values for pathological parameters; determine a pathological cancer stage based on the final predicted values for the pathological parameters; and generate a binary surgical recommendation for elective neck dissection, the recommendation being associated with a confidence index and based on the pathological cancer stage and the final predicted values.

### BRIEF DESCRIPTION OF THE DRAWINGS

Embodiments herein are illustrated in the accompanying drawings, throughout which like reference letters indicate corresponding parts in the various figures. The example embodiments herein will be better understood from the following description with reference to the drawings, in which:

FIG. 1 illustrates a block diagram for predicting lymph node status in patients with cancer using pre-operative parameters and machine learning, in accordance with an implementation of the present invention.

FIG. 2 illustrates a flowchart showing a method for predicting lymph node status in patients with cancer using pre-operative parameters and machine learning, in accordance with an embodiment of the present invention.

FIG. 3 illustrates a flowchart showing a method of determining Depth of Invasion (DOI), in accordance with an exemplary embodiment of the present invention.

FIG. 4 illustrates a flowchart showing a method of determining Neck Node Positivity prediction (PNStage), in accordance with an exemplary embodiment of the present invention.

FIG. 5 illustrates a flowchart showing an exemplary method for predicting lymph node status in patients with cancer using pre-operative parameters and machine learning, in accordance with an exemplary implementation of the present invention, in accordance with an implementation of the present invention.

### DETAILED DESCRIPTION

The embodiments herein and the various features and advantageous details thereof are explained more fully with reference to the non-limiting embodiments and detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Some embodiments of this disclosure, illustrating all its features, will now be discussed in detail. The words “enabling”, “establishing”, and other forms thereof, are intended to be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. The terms “comprises,” “comprising,” “has,” “having,” “includes” and/or “including” as used herein, specify the presence of stated features, elements, and/or components and the like, but do not preclude the presence or addition of one or more other features, elements, components and/or combinations thereof. The term “an embodiment” is to be read as “at least one embodiment.” The term “another embodiment” is to be read as “at least one other embodiment.” Although any system and methods similar or equivalent to those described herein can be used in the practice or testing of embodiments of the present disclosure, the exemplary system and methods are now described.

The disclosed embodiments are merely examples of the disclosure, which may be embodied in various forms. Various modifications to the embodiment will be readily apparent to those skilled in the art and the generic principles herein may be applied to other embodiments. However, one of ordinary skill in the art will readily recognize that the present disclosure is not intended to be limited to the embodiments described but is to be accorded the widest scope consistent with the principles and features described herein.

The detailed description set forth below in connection with the appended drawings is intended as a description of various implementations of the present disclosure and is not intended to represent the only implementations in which details of the present disclosure may be applied. Each implementation described in this disclosure is provided merely as an example or illustration, and should not necessarily be construed as preferred or advantageous over other implementations.

There is a need for a system to predict lymph node status in patients with cancer using pre-operative parameters and machine learning.

It must be understood that reference of any specific application in current disclosure, such as the automated and objective model for predicting pathological parameters by integrating radiological and clinical parameters in oncology patients, is merely provided for the ease of explanation, and should not be construed as a limiting factor for application of the methodologies described herein. Therefore, it is fairly possible for a person skilled in the art to utilize the details provided in current disclosure for any similar application.

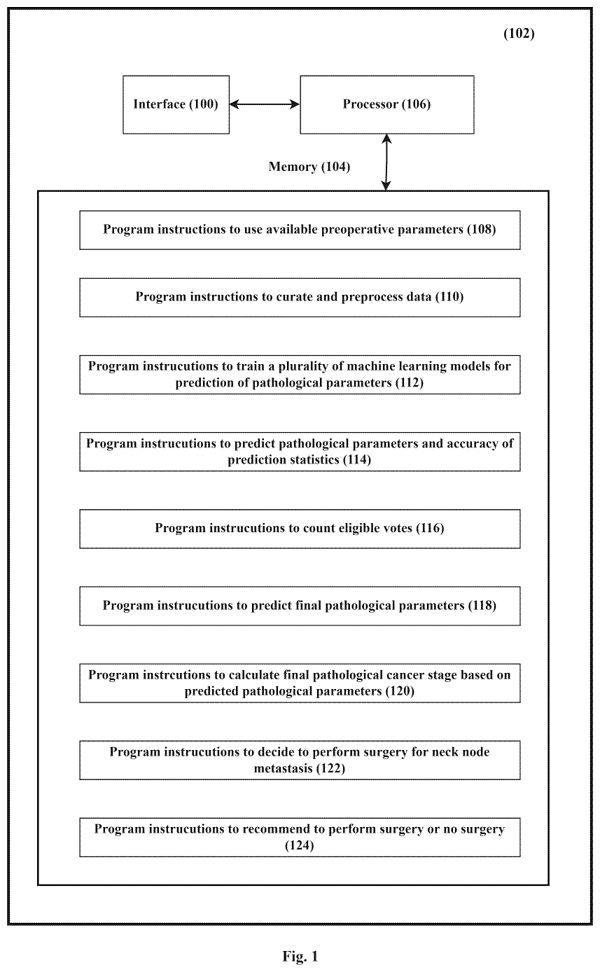

FIG. 1 illustrates a block diagram of a system 102 for predicting lymph node status in patients with cancer using pre-operative parameters and machine learning, in accordance with an implementation of the present invention. The system 102 comprises a memory 104, a processor 106, and an interface 100. The memory 104 stores program instructions for predicting pathological parameters from preoperative data using machine learning models. The instructions include acquiring preoperative parameters 108, curating and preprocessing data 110, training a plurality of machine learning models 112, predicting pathological parameters and computing prediction accuracy 114, counting eligible votes 116, determining final pathological parameters 118, calculating pathological cancer stage 120, making surgical decisions for neck node metastasis 122, and generating a final surgery recommendation 124.

The program instructions for acquiring preoperative parameters 108 enable the processor 106 to retrieve data from patient-specific clinical assessments, radiological imaging modalities (MRI, CT, ultrasound etc.), demographic details, and biochemical test results. In certain embodiments, these parameters are expanded to include a comprehensive set of non-pathological patient data configured to personalize the prediction model. This non-pathological data may comprise patient-specific behaviors such as diet and fluid intake, medication usage, smoking habits, and alcohol consumption; co-morbidities such as hypertension and diabetes; environmental factors such as geographical location and climate; and genetic data. These parameters are selected for their correlation with histopathological findings and are formatted into structured input data.

The program instructions for curating and preprocessing data 110 allow the processor 106 to perform data cleansing and enhancement operations. These operations include normalization of numerical values, imputation of missing data, removal of outliers, noise filtering, and harmonization of measurement units. Imaging data may also be aligned spatially to ensure accurate feature extraction in later stages.

The program instructions for training a plurality of machine learning models 112 enable the processor 106 to train multiple supervised learning models using labeled datasets that include known pathological parameters. The machine learning models may include, but are not limited to, Support Vector Machines (SVMs), random forest classifiers, gradient boosting machines, and deep learning neural networks. Each machine learning model may be specialized in predicting one or more pathological parameters, including but not limited to Pathological Tumor stage (pT), Pathological Nodal stage (pN), Depth of Invasion (DOI), Extranodal Extension (ENE), Lymphovascular Invasion (LVI), Perineural Invasion (PNI), and margin status.

The program instructions for predicting pathological parameters and computing prediction accuracy 114 allow the processor 106 to apply the trained models to the preprocessed patient data and generate predictions. Each model outputs predicted values along with confidence scores or probability distributions. Prediction accuracy statistics such as precision, recall, specificity, and Area Under the Curve (AUC) are computed to evaluate the reliability of each model's output.

The program instructions for counting eligible votes 116 enable the processor 106 to evaluate the set of predicted values from the plurality of machine learning models. Each prediction is treated as a “vote,” and eligibility is determined based on predefined thresholds such as minimum confidence level or historical model performance. Only eligible votes are included in the final aggregation.

The program instructions for determining final pathological parameters 118 allow the processor 106 to aggregate the eligible predictions using ensemble techniques. These include majority voting, weighted voting (based on machine learning model accuracy), or probabilistic averaging. The result is a consensus prediction for each target pathological parameter with improved reliability.

The program instructions for calculating pathological cancer stage 120 enable the processor 106 to compute the overall pathological stage of the patient based on the final predicted values of parameters such as pT and pN. The staging is performed in accordance with standard oncology guidelines, such as those defined by the American Joint Committee on Cancer (AJCC).

The program instructions for making surgical decisions for neck node metastasis 122 allow the processor 106 to assess whether the predicted nodal involvement (pN stage) indicates the presence of metastasis. If the prediction exceeds defined clinical thresholds, the system determines that neck dissection surgery should be considered.

The program instructions for generating a final surgery recommendation 124 enable the processor 106 to deliver a binary output, either surgery recommended or surgery not recommended, based on the predicted pathological parameters and the calculated cancer stage. This decision support output is communicated via the interface 100 to a clinician or surgical planning system.

In one embodiment, the processor 106 generates a confidence index associated with the surgery recommendation, enabling clinical users to assess the risk-benefit tradeoff based on model certainty.

In one embodiment, the processor 106 applies adaptive ensemble learning strategies to dynamically adjust the voting mechanism used in prediction aggregation based on patient cohort characteristics such as tumor site, stage, or demographic profile, or in response to updated training data. This dynamic adjustment enhances the system's flexibility and accuracy across varied patient populations.

In another embodiment, the system 102 supports federated learning, allowing machine learning models to be trained across multiple clinical centers without transferring patient-level data. The memory 104 stores federated learning instructions, ensuring privacy-preserving collaboration while enhancing model generalizability.

Additionally, the system 102 can be integrated with an Electronic Health Record (EHR) system, enabling automated retrieval and updating of patient-specific data. This integration facilitates real-time clinical decision support by ensuring that the system operates on current and comprehensive patient information.

In another embodiment, in order to improve clinical usability, the system includes a graphical user interface (GUI) that visually displays the predicted pathological parameters, surgical recommendation, and associated confidence index. The GUI ensures that the outputs are interpretable and actionable by clinicians.

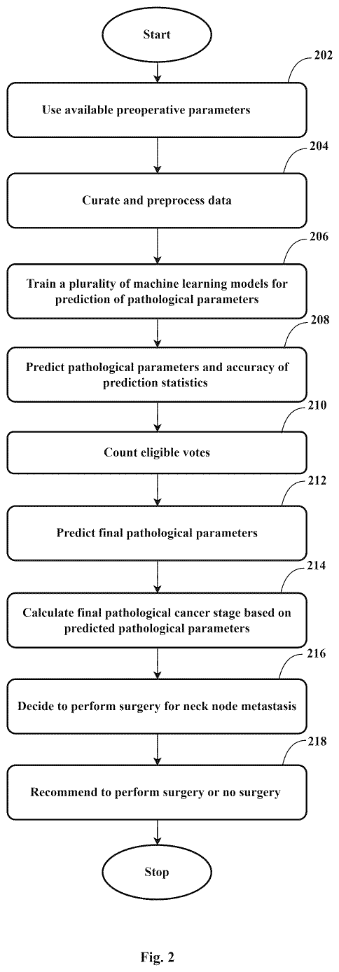

FIG. 2 illustrates a flowchart depicting a method for predicting lymph node status in patients with cancer using pre-operative parameters and machine learning, in accordance with an implementation of the present invention. At step 202, preoperative patient parameters are acquired, including demographic data, radiological imaging (e.g., MRI, CT, ultrasound), clinical examination findings, and preoperative laboratory or biopsy results. In accordance with the invention, the set of pre-operative parameters is configurable on a case-by-case basis and may be expanded to incorporate non-pathological patient data, such as patient-specific behaviors, environmental factors, co-morbidities, and genetic markers, allowing customization and personalization depending on clinical context or cancer type.

At step 204, the acquired data undergoes curation and preprocessing. This includes normalization of numerical values, imputation of missing data, noise reduction, and alignment of imaging data to ensure consistency and reliability across patient records.

At step 206, a plurality of machine learning models may be trained using the preprocessed dataset and corresponding labeled pathological outcomes. Machine learning models, including but not limited to support vector machines, random forests, gradient boosting machines, and deep neural networks, are used to learn patterns between preoperative parameter inputs and target pathological parameters.

At step 208, the trained models are applied to predict pathological parameters for a new patient. The pathological parameters include, but are not limited to, Pathological Tumor stage (pT), Pathological Nodal stage (pN), Depth of Invasion (DOI), Extranodal Extension (ENE), Lymphovascular Invasion (LVI), Perineural Invasion (PNI), and margin status. However, the algorithm is designed to be extensible, and may incorporate additional or alternative pathological and non-pathological parameters to improve predictive performance and adapt to evolving clinical protocols. Examples of non-pathological parameters include patient-specific behaviors such as smoking habits, alcohol consumption, medication usage, diet and fluid intake, exercise, and traveling; underlying conditions like hypertension and diabetes; as well as demographic and environmental factors including gender, ethnicity, geographical location, living conditions, and climate or weather at a given location. These parameters can be used to further personalize predictions for each patient. Collectively, genetic and personalized data will be used to calculate the predictions. Prediction accuracy metrics such as sensitivity, specificity, and confidence intervals may also be calculated.

At step 210, the system evaluates all model outputs and counts eligible votes from the model ensemble. Each model's prediction may be treated as a vote, and votes are considered eligible if the model's confidence level exceeds a predefined threshold or meets historical performance benchmarks.

At step 212, the system aggregates the eligible votes to derive the final predicted values of the pathological parameters. Aggregation strategies may include majority voting, weighted averaging, or probabilistic fusion depending on model performance and context.

At step 214, the final pathological cancer stage is calculated based on the aggregated predictions. This staging follows standard oncology guidelines, such as those defined by the American Joint Committee on Cancer (AJCC), and integrates predicted values for tumor size, nodal involvement, and invasiveness.

At step 216, the system evaluates the predicted pathological nodal involvement to assess the likelihood of neck node metastasis. Based on predefined clinical thresholds and risk stratification criteria, the system determines whether the surgical recommendation should be classified as surgery recommended or surgery not recommended.

At step 218, the system generates a binary recommendation for surgical planning, either surgery recommended or surgery not recommended, based on the predicted metastatic risk and the computed pathological cancer stage.

In one embodiment, the system computes a confidence score or uncertainty metric for the final surgical recommendation, enabling clinicians to evaluate the reliability of the model's output and incorporate expert judgment. The method may further include dynamically adjusting the voting mechanism based on patient cohort features. It may also involve retrieving and updating patient-specific data from Electronic Health Record (EHR) systems and generating graphical outputs via a user interface to assist clinicians in interpreting model results. In some implementations, the method incorporates federated learning frameworks to enable distributed model training across multiple centers without sharing raw patient data, thereby preserving privacy and enhancing generalizability.

In some embodiments, patient-specific data may be securely stored in a cloud-based environment, enabling real-time access, scalable computation, and remote model execution while adhering to data privacy regulations and institutional access controls.

The method is illustrated in FIG. 2 as a collection of operations in a logical flow graph representing a sequence of operations that can be implemented in hardware, software, firmware or a combination thereof.

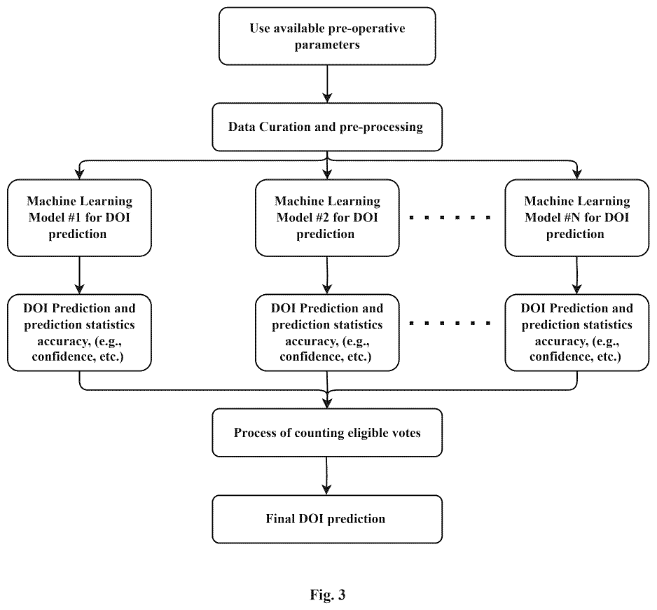

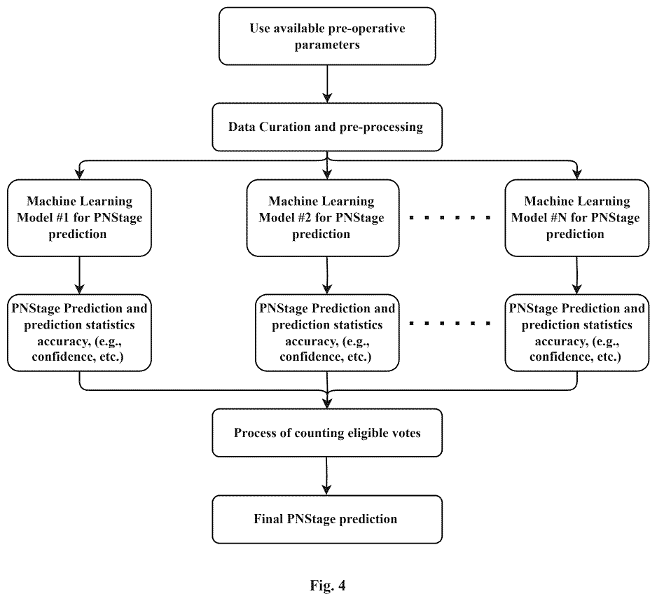

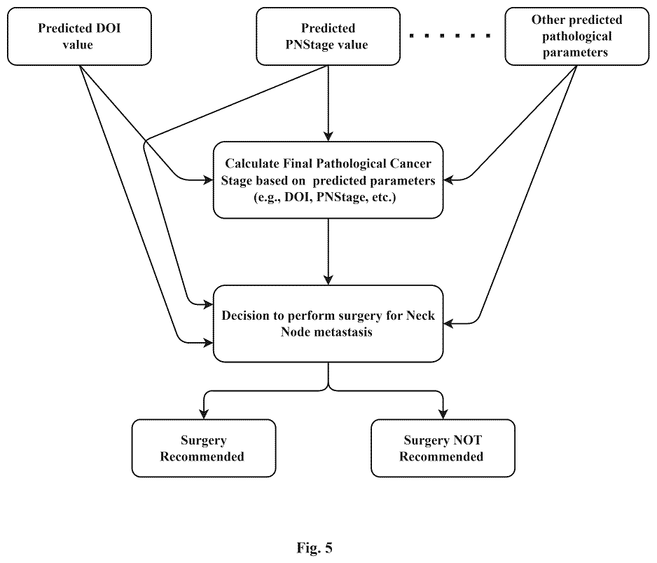

A method for predicting lymph node status using preoperative parameters and machine learning, in an exemplary implementation such as tongue cancer, is illustrated in FIGS. 3, 4, and 5. The method includes acquiring patient-specific preoperative data, preprocessing it to create structured inputs, and applying multiple trained machine learning models to predict pathological parameters. The predicted pathological parameters are evaluated for confidence, and eligible predictions are aggregated to generate final pathological values. Based on the final pathological values, the cancer stage is computed, nodal involvement is assessed, and a binary surgical recommendation, either “surgery recommended” or “surgery not recommended,” may be provided.

A main advantage of the present invention is that it enables early and accurate prediction of pathological parameters such as Pathological Tumor stage (pT), Pathological Nodal stage (pN), Depth of Invasion (DOI), Extranodal Extension (ENE), Lymphovascular Invasion (LVI), Perineural Invasion (PNI), and margin status using only preoperative data, eliminating the need for invasive or post-surgical histopathological analysis. Another advantage of the present invention is that it utilizes a plurality of machine learning models with a voting-based consensus mechanism to enhance prediction robustness and reduce bias.

Yet another advantage of the present invention is that it calculates prediction accuracy statistics and confidence scores for each parameter, allowing clinicians to assess the reliability of each output and make more informed decisions.

Still another advantage of the present invention is that it facilitates automated surgical decision-making by predicting neck node metastasis and recommending whether surgery should be performed, thereby enhancing precision oncology workflows.

Yet another advantage of the present invention is that it is scalable and modular, making it adaptable to integration into electronic health record systems and existing clinical workflows without significant infrastructure changes.

Yet another advantage of the present invention is that it supports adaptive learning through mechanisms like transfer learning and federated learning, allowing the model to evolve with new patient cohorts and across different institutions while preserving data privacy. A further advantage is the enhanced personalization achieved by incorporating non-pathological and genetic data, which allows the system to generate predictions tailored to the unique risk profile of an individual patient, rather than relying on generalized population data. Yet another advantage of the present invention is that it reduces healthcare costs and patient risks by minimizing unnecessary surgeries and enabling non-invasive, data-driven cancer staging and treatment planning.

The term “software” as used herein is intended to encompass such instructions stored in storage medium such as RAM, a hard disk, optical disk, cloud hosted or so forth, and is also intended to encompass so-called “firmware” that is software stored on a ROM or so forth. Such software may be organized in various ways, and may include software components organized as libraries, Internet-based programs stored on a remote server or so forth, source code, interpretive code, object code, directly executable code, and so forth. It is contemplated that the software may invoke system-level code or calls to other software residing on server or other location to perform certain functions.

An embodiment of the invention may be an article of manufacture in which a machine-readable medium (such as microelectronic memory) has stored thereon instructions which program one or more data processing components (generically referred to here as a “processor”) to perform the operations described above. In other embodiments, some of these operations might be performed by specific hardware components that contain hardwired logic (e.g., dedicated digital filter blocks and state machines). Those operations might alternatively be performed by any combination of programmed data processing components and fixed hardwired circuit components.

As used in the present specification, the term “machine learning” refers broadly to an artificial intelligence technique in which a computer's behavior evolves based on empirical data. In some cases, input empirical data may come from databases and yield patterns or predictions thought to be features of the mechanism that generated the data. Further, a major focus of artificial intelligence is the design of algorithms that recognize complex patterns and makes intelligent decisions based on input data. Artificial Intelligence may incorporate a number of methods and techniques such as; supervised learning, unsupervised learning, reinforcement learning, multivariate analysis, case-based reasoning, backpropagation, and transduction.

A processor may include one or more general purpose processors (e.g., INTEL® or Advanced Micro Devices® (AMD) microprocessors) and/or one or more special purpose processors (e.g., digital signal processors or Xilinx® System On Chip (SOC) Field Programmable Gate Array (FPGA) processor), MIPS/ARM class processor, a microprocessor, a digital signal processor, an application specific integrated circuit, a microcontroller, a state machine, or any type of programmable logic array.

A memory may include but is not limited to, non-transitory machine-readable storage devices such as hard drives, magnetic tape, floppy diskettes, optical disks, Compact Disc Read-Only Memories (CD-ROMs), and magnetooptical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

Any combination of the above features and functionalities may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set as claimed in claims that issue from this application, in the specific form in which such claims issue, including any subsequent correction.

In the above description, for purposes of explanation, numerous specific details are set forth in order to provide a thorough understanding of the present systems and methods. It will be apparent the systems and methods may be practiced without these specific details. Reference in the specification to “an example” or similar language means that a particular feature, structure, or characteristic described in connection with that example is included as described, but may not be included in other examples.

The foregoing description of the specific embodiments will so fully reveal the general nature of the embodiments herein that others can, by applying current knowledge, readily configure and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A system for predicting lymph node metastasis status in patients with cancer using pre-operative parameters and machine learning, the system comprising:
a memory 104 storing program instructions;
a processor 106 configured to execute the instructions to:
acquire a set of pre-operative patient parameters, the parameters comprising clinical data, radiological data, and non-pathological patient data for personalizing a prediction;
preprocess the pre-operative patient parameters to generate structured patient-specific data;
apply a plurality of machine learning models to the structured patient-specific data, wherein each machine learning model is trained on historical pathological outcomes, to generate pathological predictions;
aggregate the pathological predictions from the plurality of machine learning models using a voting mechanism to compute final predicted values for pathological parameters;
determine a pathological cancer stage based on the final predicted values for the pathological parameters; and
generate a binary surgical recommendation for elective neck dissection, the recommendation being associated with a confidence index and based on the pathological cancer stage and the final predicted values.

2. The system of claim 1, wherein the non-pathological patient data comprises at least one of patient-specific behaviors, co-morbidities, demographic attributes, environmental factors, or genetic data.

3. The system of claim 2, wherein the patient-specific behaviors comprise at least one of smoking habits, alcohol consumption, medication usage, or diet and fluid intake, and wherein the environmental factors comprise at least one of geographical location, living conditions, or climate.

4. The system of claim 1, wherein the processor is further configured to dynamically adjust the voting mechanism based on characteristics of a patient cohort.

5. The system of claim 1, wherein the confidence index associated with the binary surgical recommendation is based on at least one of a prediction certainty of individual models, a variance among model predictions, or a historical model accuracy.

6. The system of claim 1, wherein the binary surgical recommendation comprises either “surgery recommended” or “surgery not recommended” for elective neck dissection.

7. The system of claim 1, wherein the memory further stores federated learning instructions to enable multi-center model training without transferring patient-level data.

8. The system of claim 1, wherein the system is integrated with an electronic health record system to retrieve and update the pre-operative patient parameters.

9. The system of claim 1, wherein the processor is further configured to provide visualizations of predicted pathological parameters and binary surgical recommendations via a graphical user interface.

10. A computer-implemented method for predicting lymph node metastasis status in patients with cancer, the method comprising:
acquiring a set of pre-operative patient parameters, the parameters comprising clinical data, radiological data, and non-pathological patient data for personalizing a prediction;
preprocessing the pre-operative patient parameters to generate structured patient-specific data;
applying a plurality of machine learning models to the structured patient-specific data, wherein each machine learning model is trained on historical pathological outcomes, to generate pathological predictions;
aggregating the pathological predictions from the plurality of machine learning models using a voting mechanism to compute final predicted values for pathological parameters;
determining a pathological cancer stage based on the final predicted values for the pathological parameters; and
generating a binary surgical recommendation for elective neck dissection, the recommendation being associated with a confidence index and based on the pathological cancer stage and the final predicted values.

11. The method of claim 10, wherein the non-pathological patient data comprises at least one of patient-specific behaviors, co-morbidities, demographic attributes, environmental factors, or genetic data.

12. The method of claim 10, further comprising dynamically adjusting the voting mechanism based on characteristics of a patient cohort.

13. The method of claim 10, wherein the confidence index associated with the binary surgical recommendation is based on at least one of a prediction certainty of individual models, a variance among model predictions, or a historical model accuracy.

14. The method of claim 10, wherein the binary surgical recommendation comprises either “surgery recommended” or “surgery not recommended” for elective neck dissection.

15. The method of claim 10, further comprising training the plurality of machine learning models using a federated learning framework across multiple centers without transferring patient-level data.

16. The method of claim 10, further comprising retrieving and updating the pre-operative patient parameters from an electronic health record system.

17. The method of claim 10, further comprising generating visualizations of the predicted pathological parameters and the binary surgical recommendation via a graphical user interface.
